(19) United States
(12) Patent Application Publication
(10) Pub. No.: US 2014/0057256 A1
(43) Pub. Date: Feb. 27, 2014

(54) HAIRPIN-TYPE PROBE FOR DETECTING TARGET MATERIAL AND METHOD FOR DETECTING TARGET MATERIAL USING THE SAME

(71) Applicants: KOREA ADVANCED INSTITUTE OF SCIENCE AND TECHNOLOGY, (US); LG ELECTRONICS INC., Seoul (KR)

(72) Inventors: Bongchu SHIM, Seoul (KR); Hyungyu PARK, Yuseong-gu (KR); Songyi BAEK, Yuseong-gu (KR); Byoungyeon WON, Yuseong-gu (KR); Jitae KIM, Seoul (KR); Seongmoon CHO, Seoul (KR)

(73) Assignees: KOREA ADVANCED INSTITUTE OF SCIENCE AND TECHNOLOGY, Yuseong-gu (KR); LG ELECTRONICS INC., Seoul (KR)

(21) Appl. No.: 13/892,820

(22) Filed: May 13, 2013

(30) Foreign Application Priority Data
Aug. 21, 2012 (KR) 10-2012-0091222

(51) Int. Cl. C12Q 1/68

(52) U.S. Cl. CPC C12Q 1/689
USPC 435/6.11; 536/24.32

(57) ABSTRACT

The present invention relates to a hairpin-type probe for detecting a target substance and a method for detecting a target substance using the probe. The hairpin-type probe comprises a loop comprising a target substance recognition site, and a stem comprising an aptamer having an electrochemical signaling material bound thereto. The hairpin structure is broken when it is hybridized to the target substance, and thus the signaling material is separated from the aptamer and can freely move to the electrode. Based on the change in the electrochemical signal generated from the signaling material, the amount of the target substance can be accurately detected in real-time.

### BACKGROUND OF THE INVENTION

1. Field of the Invention

The present invention relates to a probe for detecting a target substance, and more particularly to a hairpin-type probe for detecting a target substance, which can accurately detect the amount of the target substance in real-time based on a change in an electrochemical signal which is generated by the signaling material when the probe is hybridized to the target substance, and to an electrochemical method for detecting a target substance using the hairpin-type probe.

2. Description of the Prior Art

Nucleic acid amplification technology has been developed to quantitative nucleic acid amplification technology since 1990s, and the market size thereof is increasing rapidly. Conventional PCR technology has a shortcoming in that the initial concentration of a target nucleic acid before an amplification process cannot be predicted, but quantitative amplification technology overcomes this shortcoming and makes it possible to accurately diagnose the initial concentration of the target nucleic acid. Typically, real-time PCR techniques including a quantitative polymerase chain reaction (qPCR) employing the non-specific fluorescent SYBR green and a nucleotide sequence-specific TaqMan probe has been extensively used, and many studies thereon have been reported. Such real-time PCR techniques have recently been recognized as the most potent techniques in the field of gene analysis and have been extensively used in the field of nucleic acid analysis. The real-time PCR techniques have demonstrated their value as they were used for the diagnosis of swine flu (H1N1) virus infection which recently prevailed worldwide.

However, current real-time PCR methods require large and expensive analysis systems, expensive fluorescent substances, and great professional skills. For this reason, most diagnostic processes employing the real-time PCR methods can be performed only by university/general hospitals or diagnostic institutions, which have technical equipment and experts, and thus large amounts of time and cost are required throughout the process to from sampling to the notice of analysis results. To overcome this limitation, point-of-care testing systems (POCTs) should be provided which can also be used in small-sized hospitals or public health centers, or even in the home, and for this purpose, it is required to develop new methods making it possible to perform analysis in small-sized systems in an expensive and simple manner.

For example, studies on real-time PCR based on a simple and inexpensive electrochemical analysis method making it easy to miniaturize systems have recently been actively conducted worldwide.

However, methods reported to date have shortcomings in that it is difficult to perform multi-target analysis for simultaneously detecting several kinds of target nucleic acids in a single reaction chamber, and labeling with separate fluorescent substances (interchelators) should be performed, and expensive optical systems are required for analysis.

### SUMMARY OF THE INVENTION

Accordingly, the present invention has been made in order to solve the above-described problems, and it is an object of the present invention to provide a hairpin-type probe for detecting a target substance, which can accurately detect the amount of the target substance in real-time, and an electrochemical method for detecting a target substance using the probe.

Another object of the present invention is to provide a one-step method for detecting a nucleic acid using a hairpin-type probe having an electrochemical signaling material bound thereto, and a multi-target real-time PCR system which does not require labeling with a separate fluorescent substance.

To achieve the above objects, the present invention provides a hairpin-type probe for detecting a target substance, the probe comprising: a loop comprising a target substance recognition site; and a stem comprising an aptamer having an electrochemical signaling material bound thereto.

In the present invention, the target substance is preferably a nucleic acid.

The loop may have a hook or ring shape.

Also, the stem and the loop may be connected to each other. Preferably, the stem is connected to both ends of the loop.

Further, the target substance recognition site may be an aptamer binding to the target substance.

In the present invention, the electrochemical signaling material may be separated from the aptamer when the loop is hybridized to the target substance.

The electrochemical signaling material is preferably one or more selected from the group consisting of metal ions, nanoparticles, quantum dots, crystal violet, ferricyanide, ferrocene derivatives, ruthenium derivatives, osmium derivatives, quinine-based proteins, and daunomycin-based proteins. Among them, a silver ion (Ag⁺) or a mercury ion (Hg²⁺) is more preferably used as the electrochemical signaling material.

Furthermore, the aptamer preferably comprises a repeat sequence of C-C or T-T mismatches.

Moreover, the electrochemical signaling material may be a silver ion (Ag⁺), and the aptamer may comprise a repeat sequence of C-C mismatches.

In addition, the electrochemical signaling material may be a mercury ion (Hg²⁺), and the aptamer may comprise a repeat sequence of T-T mismatches.

Further, the electrochemical signaling material may be a lead ion (Pb²⁺), and the to aptamer may comprise a G-quadruplex sequence.

In another aspect, the present invention provides a hairpin-type probe set for detecting target substances, the probe set comprising: a first hairpin-type probe for detecting a first target substance, the first probe comprising a loop, which comprises a first target substance recognition site, and a stem comprising an aptamer having a first electrochemical signaling material bound thereto; and a second hairpin-type probe for detecting a second target substance, the second probe comprising a loop, which comprises a second target substance recognition site, and a stem comprising an aptamer having a second electrochemical signaling material bound thereto.

Herein, the first electrochemical signaling material and the second electrochemical signaling material preferably have different oxidation potentials.

In still another aspect, the present invention provides a method for detecting a target substance, the method comprising the steps of: allowing a probe for detecting the target substance to react with the target substance; and detecting an electrochemical signal generated by the reaction.

Herein, the electrochemical signal generated by the reaction is preferably generated by separation of the electrochemical signaling material of the probe from the aptamer.

Also, the step of allowing the probe to react with the target substance may comprise a step of amplifying the target substance.

The present invention also provides a method for detecting a plurality of target substances using the above-described hairpin-type probe set, the method comprising the steps of allowing the hairpin-type probe set to react with two or more kinds of target substances; and detecting different electrochemical signals generated by the reactions.

Particulars of other embodiments are incorporated in the following detailed description and the accompanying drawings.

### BRIEF DESCRIPTION OF THE DRAWINGS

The above and other objects, features and advantages of the present invention will be more clearly understood from the following detailed description taken in conjunction with the accompanying drawing, in which:

FIG. 1 is a schematic view illustrating the structure and function of a hairpin-type probe for detecting a target substance according to a preferred embodiment of the present invention;

FIG. 2 is a schematic view illustrating a method of detecting the amount of a target substance using a hairpin-type probe for detecting the target substance according to a preferred embodiment of the present invention;

FIG. 3 is a schematic view showing examples of the signaling material and stem of a hairpin-type probe for detecting a target substance according to a preferred embodiment of the present invention;

FIG. 4 is a schematic view illustrating a hairpin-type probe for detecting a target substance according to a preferred embodiment of the present invention;

FIG. 5 is a schematic view illustrating detecting various target substances using a hairpin-type probe for detecting the target substances according to a preferred embodiment of the present invention;

FIG. 6 is a graphic diagram showing that an electrochemical current signal increases with an increase in the concentration of a target substance when detecting the target substance using a hairpin-type probe for detecting the target substance according to the present invention; and

FIG. 7 is a graphic diagram showing changes in electrochemical current signals in the case of a complementary target, in which the hairpin-type probe for detecting the target substance according to the present invention has a site recognizing the target substance (nucleic acid of Chlamydia), and in the case of a noncomplementary target, in which the hairpin-type probe has no recognition site.

### DETAILED DESCRIPTION OF THE INVENTION

Although the present invention can be modified variously and have several embodiments, exemplary embodiments are illustrated in the accompanying drawings and will be described in detail in the detailed description. However, the present invention is not limited to the specific embodiments and should be construed as including all the changes, equivalents and substitutions included in the spirit and scope of the present invention. In the following description, the detailed description of related known technology will be omitted when it may obscure the subject matter of the present invention.

Terms used in this specification are used only to describe a specific embodiment and are not intended to limit the scope of the present invention. Singular expressions include plural expressions unless specified otherwise in the context thereof. In this specification, the terms “comprise”, “have”, etc., are intended to denote the existence of mentioned characteristics, numbers, steps, operations, components, parts, or combinations thereof, but do not exclude the probability of existence or addition of one or more other characteristics, numbers, steps, operations, components, parts, or combinations thereof.

The terms “first”, “second”, etc., may be used to describe various components, but the components are not limited by the terms. The terms are used only for the purpose of distinguishing a component from other components.

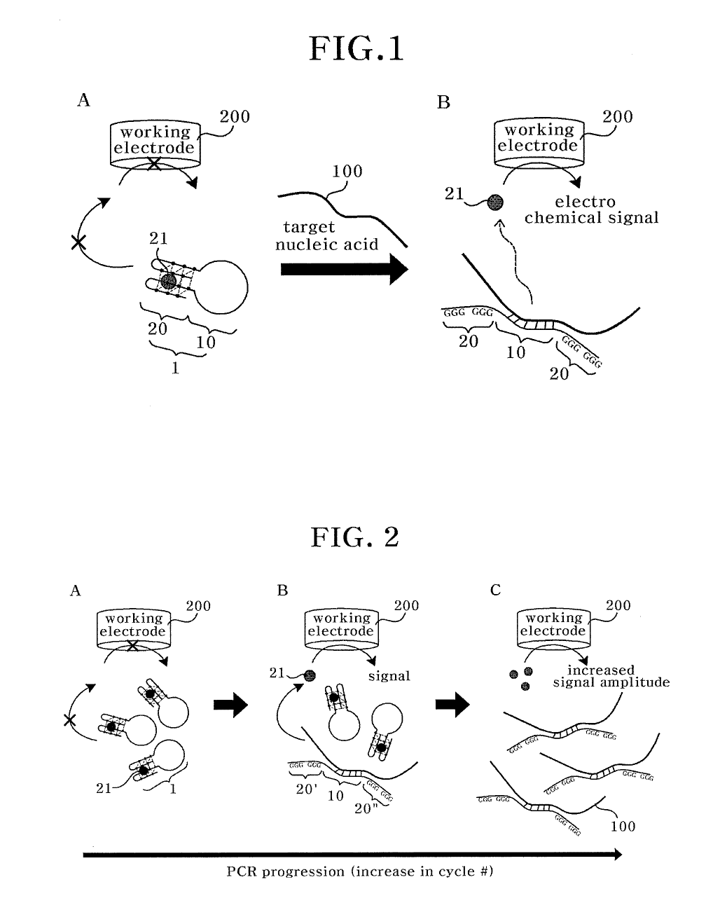

FIG. 1 is a schematic view illustrating the structure and function of a hairpin-type probe 1 for detecting a target substance according to a preferred embodiment of the present invention; FIG. 2 is a schematic view illustrating a method of detecting the amount is of a target substance 100 using a hairpin-type probe 1 for detecting the target substance according to a preferred embodiment of the present invention; and FIG. 3 is a schematic view showing examples of the signaling material 21 and stem 20 of a hairpin-type probe 1 for detecting a target substance according to a preferred embodiment of the present invention.

As shown in FIGS. 1 to 3, the probe 1 that is used to detect the target substance has a hairpin-type structure composed of a loop 10 and a stem 20. As used herein, the term “hairpin-type” refers to a form composed of the loop 10 and the stem 20, like a beacon probe. For example, as shown in FIGS. 1 and 2, the stem 20 having a two-dimensional linear structure or a three-dimensional network structure may be connected to a portion of the loop 10 having a circular structure.

The loop 10 includes a target substance recognition site and may be hybridized or bound to a target substance 100. The target substance 100 is not specifically limited, but may be a nucleic acid such as DNA or RNA. In order to detect this target substance according to the present invention, a target substance recognition site capable of recognizing the target substance 100 may be included in the whole or part of the loop 10. For example, the loop 10 preferably includes a portion of a nucleotide sequence corresponding to the target substance 100 to be detected. Although the structure of the loop 10 is not specifically limited, the loop 10 preferably has a hook or ring shape in order to facilitate the manufacture of the hairpin-type probe. The target substance 100 will be described in detail later.

The stem 20 is a portion which is connected to the loop 10 to make a hairpin-type structure. For this purpose, the stem 20 is preferably connected to both ends of the loop. One stem 20 may be connected to both ends of the loop 10. Alternatively, two or more stems 20 may also be connected to both ends of the loop 10. In the present invention, the stem 20 preferably consists of or comprises an aptamer and is characterized in that an electrochemical signaling material 21 is bound thereto. The aptamer is a biopolymer material which is in the form of single- or double-stranded DNA or RNA and is three-dimensionally bound to a target protein to inhibit protein-protein interactions. It may be used as a universal capturing agent, because it has a characteristic in that it binds to various target molecules. According to the present invention, the probe having the hair-type structure is constructed using the aptamer having such characteristics, and the electrochemical signaling material 21 is bound to the aptamer in order to detect the target substance. The method of connecting the aptamer to the loop 10 and binding the electrochemical signaling material 21 to the aptamer is not specifically limited and may be performed using various methods widely known in the art.

The electrochemical signaling material 21 is a material that generates an electrochemical signal by electrochemical voltammetry. Preferably, the electrochemical signal from the electrochemical signaling material 21 preferably changes (increases or decreases) depending on the distance of the electrochemical signaling material 21 from an electrode 200 or depending on whether attached to the electrode 200. The kind of signaling material 21 will be described in detail later.

Therefore, the probe according to the present invention may be a beacon probe comprising a stem portion, which binds specifically to the electrochemical signaling material 21, and a loop portion comprising a sequence complementary to the target to substance 100. The present invention is characterized in that, when the loop 10 is hybridized to the target substance 100, the hairpin-type structure is broken so that the electrochemical signaling material 21 is separated from the aptamer of the stem 20.

Specifically, as shown in FIG. 1, in a state in which the electrochemical signaling material 21 is bound to the stem 20 of the probe 1, the movement of the electrochemical is signaling material 21 to the surface of the electrode 200 is limited, unlike a state in which it is free from the stem 20. Thus, in this state, no electrochemical signal is generated. In other words, when the target substance 100 is not present, the signaling material 21 is bound to the aptamer so that it does not freely move to the electrode 200 in the sample solution and does not generate an electrochemical signal (see FIG. 1A). However, when the target substance 100 is present, the target substance 100 is hybridized to the sequence of the loop 10 of the probe 1, so that the structure bound to the signaling material 21 is broken and the signaling material 21 is separated. Then, the signaling material 21 is in a free state and reaches near the surface of the electrode 200, and the electrochemical signal is changed by oxidation/reduction (see FIG. 1B).

Based on this principle, the present invention uses the change in the electrochemical signal, which is obtained by electrochemical voltammetry when the amount of the target substance in the sample increases. This method is characterized in that the mass transfer of the signaling material 21 is controlled, even though the total amount of the signaling material 21 in the sample is constant. According to the present invention, a one-step analysis method for the real-time detection of the target substance is possible without labeling with a separate interchelator or an additional washing process.

For this purpose, the electrochemical signaling material 21 is a material capable of generating an electrochemical signal and includes any material that generates or does not degenerate a signal depending on the distance thereof from the electrode 200 or depending on whether attached to the electrode 200. For example, the electrochemical signaling material 21 is preferably one or more selected from the group consisting of metal ions, nanoparticles, quantum dots, crystal violet, ferricyanide, ferrocene derivatives, ruthenium derivatives, osmium derivatives, quinine-based proteins, and daunomycin-based proteins. More preferably, the electrochemical signaling material 21 is a silver ion (Ag⁺) or a mercury ion (Hg²⁺). In addition, the aptamer most preferably comprises a repeat sequence of C-C or T-T mismatches. Thus, as used herein, the term “aptamer” is meant to include not only a DNA or RNA form, but also the mismatch sequence as described above.

Specifically, the present inventors have designed the probe 1 of the present invention 1 based on the fact that the C-C mismatch sequence and the T-T mismatch sequence bind to Ag⁺ and Hg²⁺ ions, respectively, as shown in FIG. 3, and have performed experiments using the probe 1, thereby completing the present invention. When the probe 1 comprising Ag⁺ as the electrochemical signaling material 21 and a C-C mismatch sequence as the aptamer sequence is used and the target substance nucleic acid is present in a sample, Ag⁺ bound to the C-C mismatch sequence is separated to generate an electrochemical signal. When the aptamer constructed by repeating a simple sequence such as a C-C mismatch or a T-T mismatch as described is used, the signal of the target nucleic acid can be amplified, suggesting that the target nucleic acid can be analyzed with high sensitivity. In addition, it is also possible to amplify or control the magnitude of the electrochemical signal by controlling the number of C-C mismatches.

Accordingly, in the present invention, the electrochemical signaling material 21 is preferably a silver ion (Ag⁺), and the aptamer preferably comprises a repeat sequence of C-C mismatches. More preferably, the electrochemical signaling material 21 is a mercury ion (Hg²⁺), and the aptamer comprises a repeat sequence of T-T mismatches. In addition, to it is also possible that a lead ion (Pb²⁺) is used as the electrochemical signaling material 21 and that the aptamer comprises a G-quadruplex sequence binding specifically to the lead ion.

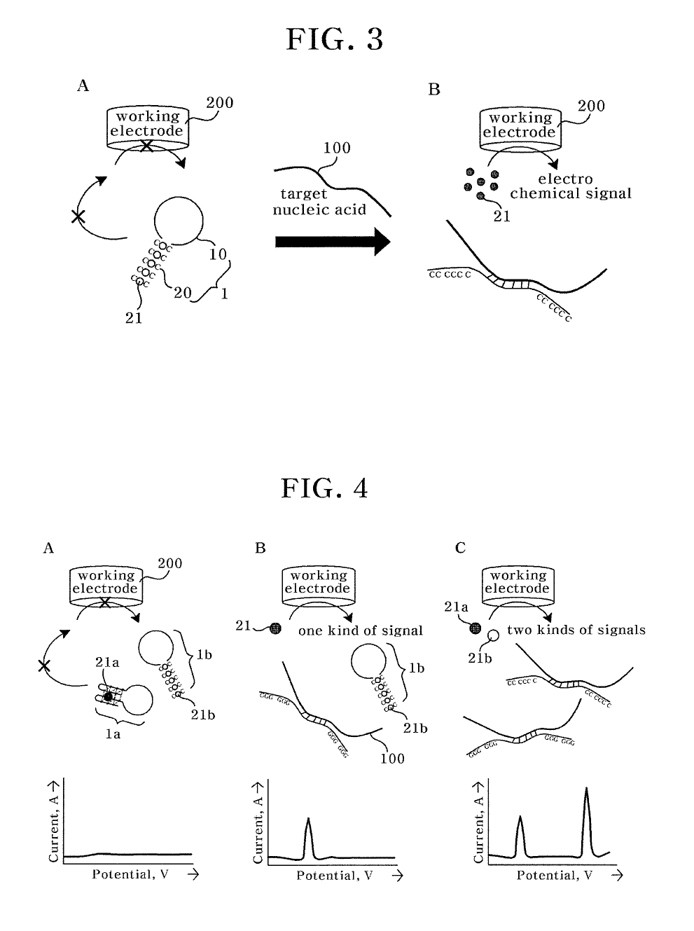

FIG. 4 is a schematic view illustrating a set of hairpin-type probes 1 for detecting target substances according to a preferred embodiment of the present invention. The is probe set shown in FIG. 4 comprises two or more kinds of probes 1 having different target substance recognition sites and electrochemical signaling materials 21.

Specifically, the present invention may provide a hairpin-type probe set for detecting target substances, which comprises: a first hairpin-type probe 1a for detecting a first target substance, the probe 1a being composed of a loop comprising a first target substance recognition site and a stem comprising an aptamer having a first electrochemical signaling material 21a bound thereto; and a second hairpin-type probe 1b for detecting a second target substance, the probe 1b being composed of a loop comprising a second target substance recognition site and a stem comprising an aptamer having a second electrochemical signaling material 21b bound thereto.

FIG. 4A shows multi-target nucleic acid analysis and multi-target real-time PCR, which are performed using several kinds of probes 1a and 1b which bind to different signaling materials 21a and 21b at different positions. The probes 1a and 1b bind to different target nucleic acids to generate different signals. Specifically, when no target nucleic acid is present in a sample in multi-target PCR for simultaneously detecting two different kinds of target nucleic acids, no electrochemical signal is generated, because two kinds of signaling materials 21a and 21b are all bound to the aptamers so that they cannot be freely diffused to the surface of the electrode 200. As shown in FIG. 4B, when only one of two kinds of target nucleic acids is present in a sample, only the one target nucleic acid is amplified and hybridized to the probe 1a, and thus only the signaling material 21a bound to the corresponding probe 1a can be freely diffused to generate a signal. On the other hand, in the probe 1b corresponding to the other target nucleic acid which is not amplified due to the absence of the target nucleic acid, the signaling material 21b corresponding thereto is maintained in a state bound to the aptamer, and thus no signal is generated. As shown in FIG. 4C, when two kinds of target nucleic acids are present in a sample, all the target nucleic acids are amplified and all the signaling materials 21a and 21b can be freely diffused, and thus two different signals are simultaneously generated.

As described above, when several kinds of probes 1a and 1b, which comprise different signaling materials 21a and 21b generating different electrochemical signals, and different aptamer sequences to which the signaling materials can be bound, are used according to the present invention, it is possible to realize a multi-target detection system for simultaneously detecting several kinds of target nucleic acids in a single reaction chamber. In this case, because different signaling materials 21a and 21b are bound to the probes 1a and 1b corresponding to different target nucleic acids, whether a specific nucleic acid is present in a sample can be determined based on the kinds of signaling materials 21a and 21b.

In the present invention, metals that are used as the signaling materials 21 are advantageously in ionic forms. Further, the metals that are used as the signaling materials 21 preferably have different oxidation potentials and may be selected from among Bi, Cd, Cu, Ga, Ge, In, Ni, Pb, Sb, Sn, Ti, Zn, Hg, Au, Ag, Pt and the like.

Analysis in the present invention is preferably performed by electrochemical stripping analysis, and additives such as mercury (Hg), bismuth (Bi) or galinstan (a mixture of gallium, indium and tin) may also be used to increase signal sensitivity.

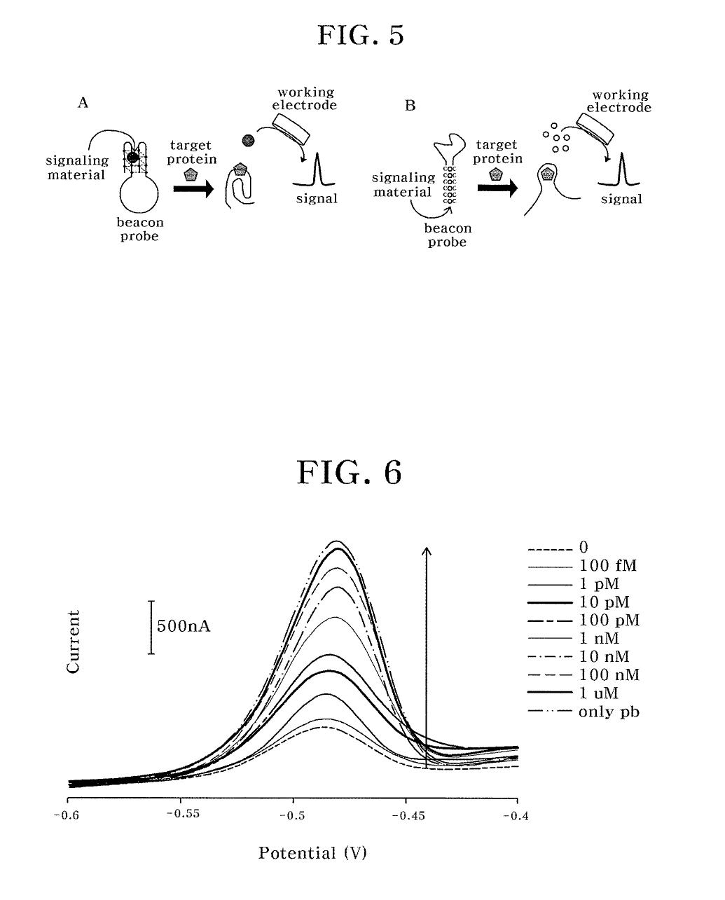

FIG. 5 is a schematic view illustrating detecting various target substances 100 using the hairpin-type probe 1 for detecting the target substance according to a preferred embodiment of the present invention. According to the present invention, not only nucleic acids, but also small molecules, proteins, cells and the like can be detected based on the nucleic acid detection method that uses the probe 1 binding to the electrochemical signaling material 21 as described above.

In other words, the loop 10 comprising an aptamer sequence, which binds specifically to a small molecule, a protein, a cell or the like, in place of a sequence complementary to a target nucleic acid, is used, the target substance 100 binds to the aptamer sequence of the loop 100 while the electrochemical signaling material becomes free to generate electrochemical signals, and thus various target substances 100 can be detected.

For this, the target substance recognition site in the loop 10 of the present invention is preferably an aptamer binding to the target substance.

In another embodiment, the present invention provides a method for detecting a target substance using the hairpin-type probe, the method comprising the steps of: allowing the hairpin-type probe 1 for detecting the target substance to react with the target substance 100; and detecting an electrochemical signal generated by the reaction.

The method for allowing the probe 1 to react with the target substance 100 is not specifically limited. For example, the probe 1 of the present invention together with a signaling material is added to a reaction chamber comprising a working electrode, a reference electrode and a counter electrode. A working electrode 200 on which an electrochemical reaction occurs may be made of a material, such as gold (Au), silver (Ag), platinum (Pt) or carbon (C), depending on the kind of signaling material. Then, a PCR reaction solution (polymerase, primers, and dNTPs) containing the target substance 100 is introduced into the reaction chamber, and PCR is performed.

Then, an electrochemical signal is measured at a time point when the annealing step of the denaturation, annealing and polymerization or extension steps of the PCR reaction is completed. When the target substance 100 is present in the sample and the amount of the target substance 100 increases as the PCR reaction progresses, the target substance 100 is hybridized to the probe 1 of the present invention, and the signaling material 21 bound to the aptamer is freely separated, resulting in an increase in the magnitude of the electrochemical signal.

In the present invention, the step of allowing the probe 1 to react with the target substance 100 may comprise a step of amplifying the target substance 100. In other words, the method of the present invention may be an electrochemical real-time PCR method in which PCR amplification and signal measurement are simultaneously performed based on the above-described detection method, and the amplification of the target nucleic acid is monitored in real-time by electrochemical analysis.

In addition, the present invention also provides a method for detecting a plurality of target substances using a hairpin-type probe set, the method comprising the steps of: allowing a set of the hairpin-type probes 1 for detecting the target substances to react with two or more kinds of target substances 10; and detecting different electrochemical signals generated by the reactions.

Specifically, the method of the present invention may also be an electrochemical multi-target PCR method in which various kinds of metal ions having different characteristic oxidation potentials are used as the signaling materials 21a and 21b, and signals from the signaling materials are simultaneously measured, and the amplification of various kinds of target nucleic acids is monitored in real-time by electrochemical analysis.

As described above, when a set of the probes 1 capable of binding to different metals depending on various kinds of target nucleic acids is used, it is possible to realize a multi-target real-time PCR system in a single reaction chamber. The presence or absence of various kinds of target nucleic acids can be monitored in real-time at the same time with PCR amplification by determining whether various kinds of electrochemical signals generated from different metals increase.

Hereinafter, the present invention will be described in further detail with reference to examples. It is to be understood, however, that these examples are for illustrative purposes only and are not intended to limit the scope of the present invention.

### Example 1

### One-Step Method for Electrochemical Detection of Nucleic Acid

According to the present invention as described above, the presence or concentration of a target nucleic acid in a sample was detected.

First, the nucleic acid of Chlamydia was selected as a target nucleic acid. A loop portion which can be hybridized to the target nucleic acid was constructed, and a stem portion was constructed using a G-quadruplex type aptamer sequence capable of binding to a lead ion (Pb²⁺) serving as a signaling material, thereby synthesizing a molecular beacon probe (GGG TAG GGT TTA AAA GGG ATT GCA GCT TGG GTT GGG).

The beacon probe and the signaling material were added to a reaction solution, and a glassy carbon working electrode, an Ag/AgCl reference electrode and a Pt counter electrode were placed in a reaction chamber. The target nucleic acid was added to the reaction solution at a concentration ranging from 1 pM to 1 μM and was allowed to react for 30 minutes, followed by anodic stripping analysis. Herein, the reaction time of 30 minutes is a time for ensuring a sufficient reaction, and thus the reaction time can be reduced depending on applications. The electrochemical signal from the signaling material was measured under the following conditions: 2 min accumulation at −0.8 V; and subsequent square-wave stripping from −0.8 to −0.2 V with step potential of 2 mV, amplitude of 25 mV, and frequency of 25 Hz.

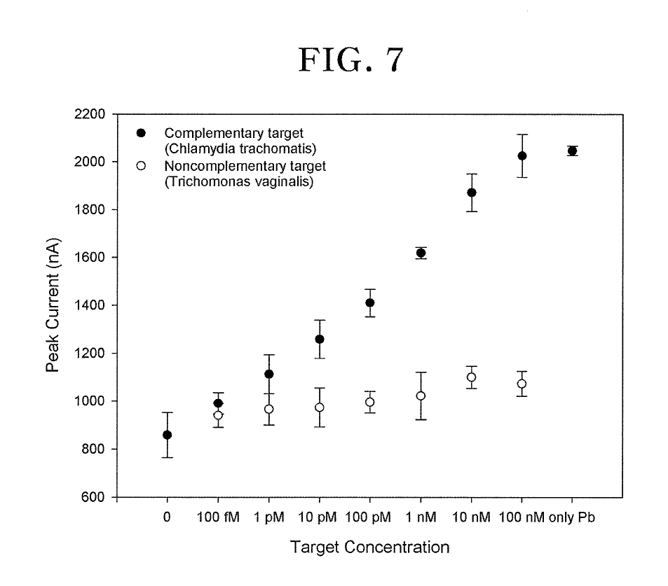

The results of the measurement are shown in FIGS. 6 and 7.

FIG. 6 is a graphic diagram showing that an electrochemical current signal increases with an increase in the concentration of a target substance when detecting the target substance using the hairpin-type probe for detecting the target substance according to the present invention. As can be seen therein, the electrochemical current signal increased as the concentration of the target nucleic acid increased.

FIG. 7 is a graphic diagram showing changes in electrochemical current signals in the case of a complementary target, in which the hairpin-type probe for detecting the target substance according to the present invention has a site recognizing the target substance (nucleic acid of Chlamydia), and in the case of a noncomplementary target, in which the hairpin-type probe has no recognition site. As can be seen therein, when the hairpin-type probe for detecting the target substance had the site recognizing the target substance, the amplitude of the electrochemical current signal increased as the concentration of the target nucleic acid increased.

As described above, the hairpin structure according to the present invention is broken when it is hybridized to the target substance, and thus the signaling material is separated from the aptamer and can freely move to the electrode. Based on the change in the electrochemical signal generated from the signaling material, the amount of the target substance can be accurately detected in real-time.

In addition, the present invention can also provide a one-step method for detecting a nucleic acid using the hairpin-type probe having an electrochemical signaling material bound thereto, and a multi-target real-time PCR system which does not require labeling with a separate fluorescent substance (interchelator).

Although the preferred embodiments of the present invention have been described for illustrative purposes, those skilled in the art will appreciate that various modifications, additions and substitutions are possible, without departing from the scope and spirit of the invention as disclosed in the accompanying claims.

What is claimed is:

1. A hairpin-type probe for detecting a target substance, the probe comprising:
a loop comprising a target substance recognition site; and
a stem comprising an aptamer having an electrochemical signaling material bound thereto.

2. The hairpin-type probe of claim 1, wherein the target substance is a nucleic acid.

3. The hairpin-type probe of claim 1, wherein the loop has a hook or ring shape.

4. The hairpin-type probe of claim 1, wherein the stem and the loop are connected to each other.

5. The hairpin-type probe of claim 1, wherein the stem is connected to both ends of the loop.

6. The hairpin-type probe of claim 1, wherein the target substance recognition site is an aptamer binding to the target substance.

7. The hairpin-type probe of claim 1, wherein the electrochemical signaling material is separated from the aptamer when the loop is hybridized to the target substance.

8. The hairpin-type probe of claim 1, wherein the electrochemical signaling material is one or more selected from the group consisting of metal ions, nanoparticles, quantum dots, crystal violet, ferricyanide, ferrocene derivatives, ruthenium derivatives, osmium derivatives, quinine-based proteins, and daunomycin-based proteins.

9. The hairpin-type probe of claim 1, wherein the electrochemical signaling material is a silver ion (Ag⁺) or a mercury ion (Hg²⁺).

10. The hairpin-type probe of claim 1, wherein the aptamer comprises a repeat sequence of C-C or T-T mismatches.

11. The hairpin-type probe of claim 1, wherein the electrochemical signaling material is a silver ion (Ag⁺), and the aptamer comprises a repeat sequence of C-C mismatches.

12. The hairpin-type probe of claim 1, wherein the electrochemical signaling material is a mercury ion (Hg²⁺), and the aptamer comprises a repeat sequence of T-T mismatches.

13. The hairpin-type probe of claim 1, wherein the electrochemical signaling material is a lead ion (Pb²⁺), and the aptamer comprises a G-quadruplex sequence.

14. A hairpin-type probe set for detecting target substances, the probe set comprising:
a first hairpin-type probe for detecting a first target substance, the first probe comprising a loop, which comprises a first target substance recognition site, and a stem comprising an aptamer having a first electrochemical signaling material bound thereto; and
a second hairpin-type probe for detecting a second target substance, the second probe comprising a loop, which comprises a second target substance recognition site, and a stem comprising an aptamer having a second electrochemical signaling material bound thereto.

15. The hairpin-type probe set of claim 14, wherein the first electrochemical signaling material and the second electrochemical signaling material have different oxidation potentials.

16. A method for detecting a target substance, the method comprising the steps of:
allowing a probe for detecting the target substance to react with the target substance; and
detecting an electrochemical signal generated by the reaction.

17. The method of claim 16, wherein the electrochemical signal generated by the reaction is generated by separation of the electrochemical signaling material of the probe from the aptamer.

18. The method of claim 16, wherein the step of allowing the probe to react with the target substance comprises a step of amplifying the target substance.

19. A method for detecting a plurality of target substances using the hairpin-type probe set of claim 14, the method comprising the steps of:
allowing the hairpin-type probe set to react with two or more kinds of target substances; and
detecting different electrochemical signals generated by the reactions.
